(19) United States
(12) Patent Application Publication
(10) Pub. No.: US 2014/0100627 A1
(43) Pub. Date: Apr. 10, 2014

(54) LEADLESS INTRA-CARDIAC MEDICAL DEVICE WITH INTEGRATED L-C RESONANT CIRCUIT PRESSURE SENSOR

(71) Applicant: PACESETTER, INC., Sylmar, CA (US)

(72) Inventor: Xiaoyi Min, Camarillo, CA (US)

(73) Assignee: PACESETTER, INC., Sylmar, CA (US)

(21) Appl. No.: 13/647,300

(22) Filed: Oct. 8, 2012

(51) Int. Cl. A61N 1/37

(52) U.S. Cl. 607/32

(57) ABSTRACT

A leadless intra-cardiac medical device comprises an integrated L-C resonant circuit pressure sensor. In some embodiments, the pressure sensor comprises a passive sensor that measures pressure in response to an externally generated excitation signal. In some embodiments, the pressure sensor comprises an active sensor that measures pressure in response to an internally generated excitation signal.

### TECHNICAL FIELD

This application relates generally to implantable medical devices and, more specifically, but not exclusively to a leadless intra-cardiac medical device with integrated L-C resonant circuit pressure sensor.

### BACKGROUND

When a person's heart does not function normally due to, for example, a genetic or acquired condition, various treatments may be prescribed to correct or compensate for the condition. For example, pharmaceutical therapy may be prescribed for a patient or a pacemaker or similar device may be implanted in the patient to improve the function of the patient's heart.

In conjunction with such therapy, it may be desirable to detect conditions in or apply therapy to one or more chambers of the heart. For example, the health of many patients who have had some form of heart failure (e.g., a heart attack) may deteriorate over time due to progressive failure of the heart.

Heart failure is a debilitating disease in which abnormal function of a patient's heart leads to inadequate blood flow to the patient's body. While a heart failure patient may not suffer debilitating symptoms immediately, with few exceptions, the disease is relentlessly progressive. Moreover, as heart failure progresses, it may become increasingly difficult to manage.

Despite current drug and device therapies, the rate of heart failure hospitalization remains high. Consequently, significant hospitalizations costs are incurred annually for heart failure patients.

Cardiac pressure monitoring has been suggested as a means for tracking heart failure progression in a patient. For example, pulmonary artery pressure has been proposed as a predictor for heart failure progression. In addition, a rise in left atrial pressure has been proposed as a potential indicator of left ventricular failure.

Consequently, it has been proposed to implant pressure sensors that will monitor cardiac pressure in various chambers. For example, it has been proposed to place a dedicated pressure sensor in a branch of the pulmonary artery for heart failure monitoring. In addition, it has proposed to incorporate pressure sensors on implantable leads to measure ventricular pressure or atrial pressure. However, these types of sensors are generally quite complicated and have a relatively high cost. In addition, there may be risks associated a dedicated implant procedure used for dedicated sensors.

Accordingly, a need exists for more effective techniques for monitoring cardiac pressure so that appropriate treatment may be readily prescribed for the patients, thereby lowering the hospitalization rate for the patients.

### SUMMARY

A summary of several sample aspects of the disclosure and embodiments of an apparatus constructed or a method practiced according to the teaching herein follows. It should be appreciated that this summary is provided for the convenience of the reader and does not wholly define the breadth of the disclosure. For convenience, one or more aspects or embodiments of the disclosure may be referred to herein simply as “some aspects” or “some embodiments.”

The disclosure relates in some aspects to a leadless intra-cardiac medical device integrated with an inductive-capacitive (L-C) resonant circuit pressure sensor. In some aspects, such a pressure sensor may be effectively employed to monitor cardiac pressure (e.g., pulmonary artery pressure, right ventricle pressure, etc.) and, therefore, treat heart failure or other cardiac conditions. For example, by monitoring changes in pressure that are indicative of heart failure, progressive heart failure may be identified and treated at a relatively early stage.

In some aspects, a leadless intra-cardiac medical device as taught herein is typically characterized by the following features: it is devoid of leads that pass out of the heart to another component, such as a pacemaker can outside of the heart; it includes electrodes that are affixed directly to the can of the device; the entire device is attached to the heart; and the device is capable of pacing and/or sensing in the chamber of the heart where it is implanted.

In some embodiments, the L-C resonant circuit pressure sensor comprises a passive sensor that measures pressure in response to an externally generated excitation signal. The passive pressure sensor comprises a resonant L-C circuit that is excited by an electromagnetic field generated by an external device. The capacitive circuit portion of the resonant circuit is flexible in some aspects such that changes in pressure at the pressure sensor (e.g., implanted in a patient's heart) distort the capacitive circuit, thereby causing changes in the capacitance of the capacitive circuit. Thus, changes in pressure at the pressure sensor are reflected by changes in the resonant frequency of the resonant circuit. Upon excitation of the resonant circuit, any changes in the resonant frequency may be detected by circuitry of the leadless intra-cardiac medical device. For example, this circuitry may processes signals received from the resonant circuit and thereby generate data representative of the pressure external to the leadless intra-cardiac medical device.

In some embodiments, the L-C resonant circuit pressure sensor comprises an active sensor that measures pressure in response to an internally generated excitation signal. For example, the L-C resonant circuit may comprise an excitation circuit that is triggered and powered by circuitry of the leadless intra-cardiac medical device. Again, the capacitive circuit portion of the resonant circuit is flexible in some aspects such that changes in pressure at the pressure sensor cause changes in the capacitance of the capacitive circuit. Accordingly, upon excitation of the L-C resonant circuit, changes in pressure at the pressure sensor are reflected by changes in the frequency of the signal induced in the resonant circuit. Data representative of the pressure external to the leadless intra-cardiac medical device may thus be generated based on these changes in frequency.

### BRIEF DESCRIPTION OF THE DRAWINGS

These and other aspects of the disclosure will be more fully understood when considered with respect to the following detailed description, the appended claims, and the accompanying drawings, wherein:

FIG. 1 is a simplified diagram of an embodiment of a leadless intra-cardiac medical device;

FIG. 2 is a simplified circuit and block diagram of an embodiment of a leadless intra-cardiac medical device;

FIG. 3 is a simplified circuit and block diagram of another embodiment of a leadless intra-cardiac medical device;

FIG. 4 is a simplified block diagram of an embodiment of a medical system illustrating communication between a leadless intra-cardiac medical device and external devices;

FIG. 5 is a simplified block diagram of another embodiment of a medical system illustrating communication between a leadless intra-cardiac medical device and an external device;

FIG. 6 is a more detailed diagram of an embodiment of a leadless intra-cardiac medical device;

FIG. 7 is a simplified diagram of an embodiment of a leadless intra-cardiac medical device implanted in a patient's heart for sensing conditions in the patient and optionally delivering therapy to the patient;

FIG. 8 is a simplified diagram of another embodiment of a leadless intra-cardiac medical device implanted in a patient's heart for sensing conditions in the patient and optionally delivering therapy to the patient;

FIG. 9 is a simplified diagram of another embodiment of a leadless intra-cardiac medical device implanted in a patient's heart for sensing conditions in the patient and optionally delivering therapy to the patient;

FIG. 10 is a simplified flowchart of an embodiment of pressure sensing operations that may be performed by, for example, circuitry of a leadless intra-cardiac medical device;

FIG. 11 is a simplified block diagram of an embodiment of communication system comprising a leadless intra-cardiac medical device and an external device; and

FIG. 12 is a simplified functional block diagram of an embodiment of a leadless intra-cardiac medical device, illustrating basic elements that may be configured to sense conditions in the patient, deliver therapy to the patient, or provide some combination thereof.

In accordance with common practice, the various features illustrated in the drawings may not be drawn to scale. Accordingly, the dimensions of the various features may be arbitrarily expanded or reduced for clarity. In addition, some of the drawings may be simplified for clarity. Thus, the drawings may not depict all of the components of a given apparatus or method. Finally, like reference numerals may be used to denote like features throughout the specification and figures.

### DETAILED DESCRIPTION

The description that follows sets forth one or more illustrative embodiments. It will be apparent that the teachings herein may be embodied in a wide variety of forms, some of which may appear to be quite different from those of the disclosed embodiments. Consequently, the specific structural and functional details disclosed herein are merely representative and do not limit the scope of the disclosure. For example, based on the teachings herein one skilled in the art should appreciate that the various structural and functional details disclosed herein may be incorporated in an embodiment independently of any other structural or functional details. Thus, an apparatus may be implemented or a method practiced using any number of the structural or functional details set forth in any disclosed embodiment(s). Also, an apparatus may be implemented or a method practiced using other structural or functional details in addition to or other than the structural or functional details set forth in any disclosed embodiment(s).

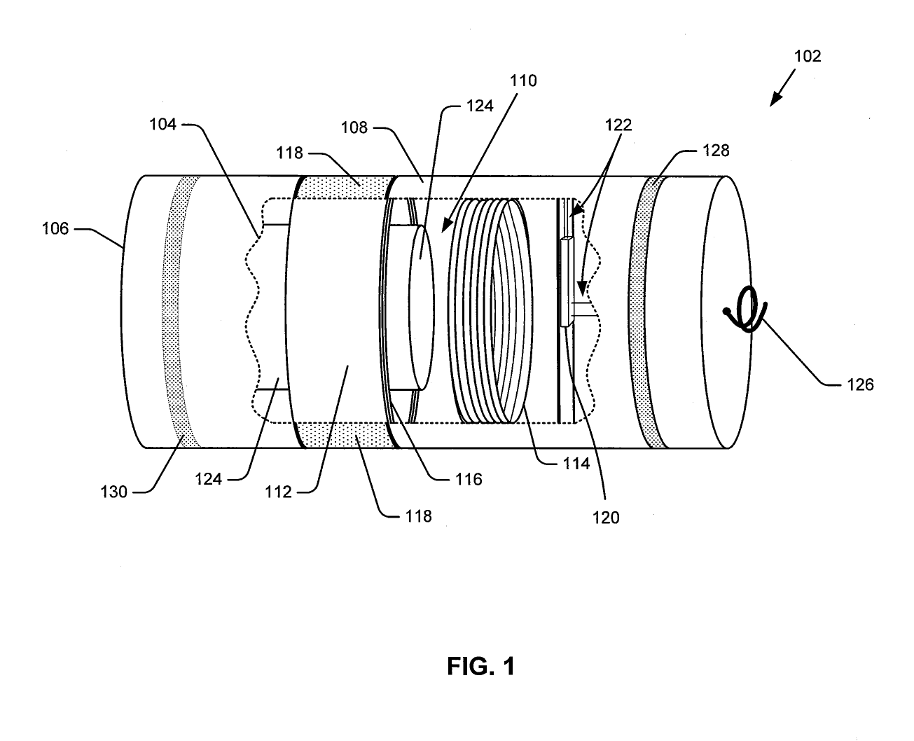

FIG. 1 illustrates, in a simplified sectional side view, an embodiment of a leadless intra-cardiac medical device 102. For purposes of illustration, the device 102 is depicted with a hypothetical opening 104 to show several interior components of the device 102. The device 102 includes a housing 106 comprising an external surface 108 and defining an interior space 110.

The device 102 comprises an L-C resonant circuit pressure sensor including a flexible capacitive circuit 112 and an inductive circuit 114. In the example of FIG. 1, the capacitive circuit 112 comprises a plurality of concentric plates 116 separated by dielectric material and the inductive circuit 114 comprises a coil inductor. Other structures may be employed for the capacitive circuit 112 and the inductive circuit 114 in other embodiments constructed according to the teachings herein.

An external surface of the L-C resonant circuit pressure sensor comprises a flexible material 118 (shown partially cut away in FIG. 1) that is located adjacent the exterior surface 108 of the housing 104. The external surface of the pressure sensor is flexible and engaged with the capacitive circuit 112 to couple pressure waves external to the device 102 to the capacitive circuit 112.

The capacitive circuit 112 includes at least one flexible component such that movement of the flexible material 118 induces movement in the flexible component(s). For example, at least one the plates and/or the dielectric material of the capacitive circuit 112 may be flexible. In this way, a change in pressure external to the device will affect the physical dimensions of the capacitive circuit (e.g., the distance between plates) and thereby induce a change in the capacitance of the capacitive circuit 112. Consequently, the resonant frequency of the L-C resonant circuit will change. As discussed in more detail below, the device 102 includes circuitry to detect this change in frequency and thereby generate data indicative of cardiac pressure external to device 102 (e.g., a change in pressure). For example, an integrated circuit 120, one or more associated electrical conductors 122, and a battery circuit 124 (comprising a battery) may be coupled to the LC-resonant circuit for detecting the operating frequency of the L-C resonant circuit and/or for exciting the L-C resonant circuit. The integrated circuit 120, the electrical conductors 122, and the battery circuit 124 are located within the interior space 110 of the housing 106.

In a typical implementation, one terminal of the inductive circuit 114 is coupled via a conductor to a plate of the capacitive circuit 112, while another terminal of the inductive circuit 114 is coupled via another conductor to another plate of the capacitive circuit 112. Thus, the inductive circuit 114 and the capacitive circuit 112 are coupled in parallel, thereby forming a resonant circuit that is capable of being excited by an externally applied electromagnetic field or an internally applied signal.

In the example of FIG. 1, the flexible material 118 is coplanar with the exterior surface 108 of the device 102. In such a case, the flexible material 118 may comprise a biocompatible material and, optionally, an insulating material (e.g., to insulate different sections of the housing 106 from one another). For example, the flexible material 118 may comprise silicone or some other flexible biocompatible material.

In other embodiments, however, the pressure sensor 104 may be located completely within the housing 106. For example, the housing 106 may be hermetically sealed and comprise a flexible material or may include a flexible section immediately above the pressure sensor. In these cases, the pressure sensor need not be biocompatible. In the above embodiments, the inductive circuit 114 is located within the housing 106, is generally inflexible, and is not coupled to an external flexible material. In this way, the inductance value of the inductive circuit 114 remains substantially fixed when the device 102 is subjected to changes in external pressure.

The device 102 also includes components for sensing cardiac signals and/or stimulating (e.g., pacing) cardiac tissue. For example, the device may operate in one or more of the following modes: VDD, DDD, VVIR, CRT, or some other suitable mode. These components may include, for example, an electrode 126 (e.g., a helical electrode), an electrode 128 (e.g., a ring electrode), one or more additional electrodes (represented by electrode 130), and other circuitry. This other circuitry may include, for example, corresponding functionality of the integrated circuit 120, one or more of the electrical conductors 122, and the battery circuit 124.

Of note, the leadless intra-cardiac medical device 102 does not include any implantable leads or any connectors for implantable leads. Instead, in some embodiments, the leadless intra-cardiac medical device 102 uses one or more of the electrodes 126-130 for directly sensing cardiac signals and/or delivering stimulation signals (e.g., pacing pulses) to cardiac tissue. It should be appreciated that different embodiments may employ a different number of electrodes (e.g., two or more electrodes) depending on the requirements of the respective deployments.

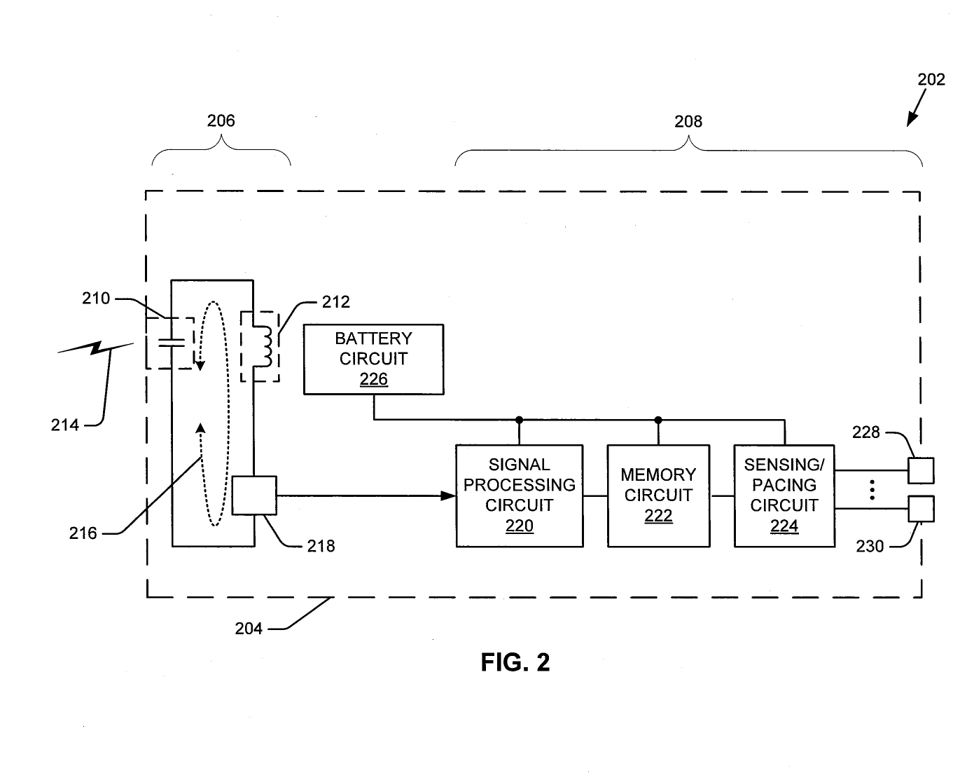

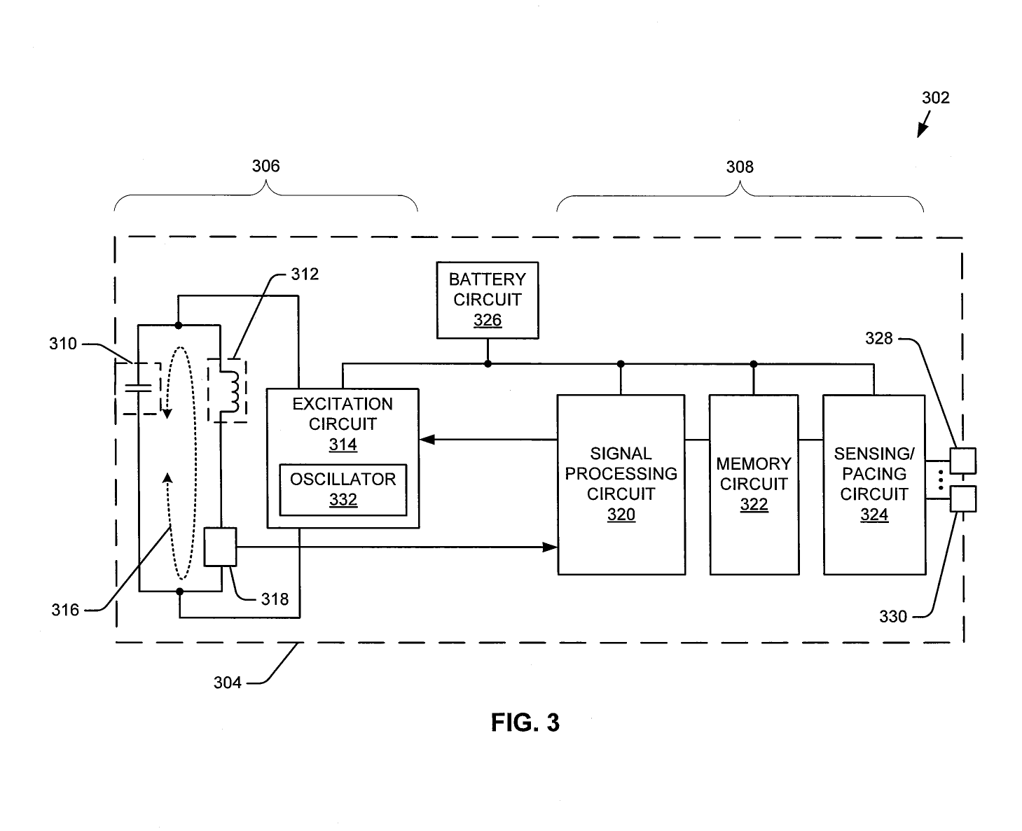

The L-C resonant circuit pressure sensor may comprise a passive sensor or an active sensor. FIGS. 2 and 3 illustrate two examples of circuitry that may be employed for these two configurations.

FIG. 2 is a simplified schematic and block diagram of an embodiment of a leadless intra-cardiac medical device 202 that comprises a passive L-C resonant circuit pressure sensor. The housing is represented by a dashed line 204. The L-C resonant circuit 206 is located at the left-most section of the device 202, while the circuit 208 to the right of the L-C resonant circuit 206 performs certain pressure sensing-related operations as well as cardiac sensing and/or pacing operations.

For purposes of illustration, the circuit 208 is depicted as including a signal processing circuit 220, a memory circuit 222, a sensing/pacing circuit 224, a battery circuit 226, and two electrodes 228 and 220. It should be appreciated that different combinations of these components may be employed in other embodiments constructed in accordance with the teachings herein.

The L-C resonant circuit 206 comprises a capacitive circuit 210 and an inductive circuit 212. The values of these components are selected to cause the L-C resonant circuit 206 to resonate at a specified nominal frequency. In this case, the L-C resonant circuit 206 is excited (e.g., induced with a signal that causes the L-C resonant circuit 206 to resonate) by an externally generated radiofrequency (RF) signal 214. Upon excitation, an oscillating signal 216 (represented, for convenience, by a dashed line) at the resonant frequency is established in the L-C resonant circuit 206. Typically, the oscillating signal 216 resulting from excitation of the L-C resonant circuit 206 will be a damping signal (i.e., decreasing in amplitude over time) since excitation signals are generally not applied to the L-C resonant circuit 206 on a continuous basis.

The capacitive circuit 210 is engaged with a flexible material adjacent an exterior surface of the housing 204 (e.g., as discussed above). Consequently, a change in pressure external to the device 202 will result in a change in the capacitance of the capacitive circuit 210. This change in capacitance, in turn, causes a change in the resonant frequency of the L-C resonant circuit 206. Thus, a change in pressure will result in a change in the frequency of the oscillating signal 216.

The oscillating signal 216 is detected by a circuit 218 (e.g., a high impedance sense amplifier, a low impedance current sensing circuit, or some other suitable circuit) and provided to the signal processing circuit 220. The signal processing circuit 220 processes the received signal to determine at least one frequency of the signal. Consequently, the signal processing circuit 220 may generate data representative of the pressure external to the device 202 based on the frequency of the received signal. For example, the generated data may comprise at least one pressure value that was determined based on the determined at least one frequency.

The signal processing circuit 220 may then store this data in the memory circuit 222 for subsequent use. For example, as discussed below, the device 202 may periodically collect data over a period of time, and send the stored data to an external device (not shown in FIG. 2) at some later point in time. As another example, one or more operating parameters (e.g., pacing parameters) of the device 202 may be adapted based on the pressure data.

To facilitate receiving the oscillating signal 216, the signal processing circuit 220 and/or the circuit 218 may comprise one or more of: a sensing circuit, an amplifier, a filter, a switching circuit, or other suitable circuits. Thus, these circuits may perform one or more of: detecting, filtering, or amplifying the oscillating signal 216.

As represented by corresponding lines in FIG. 2, the battery circuit 226 is electrically coupled to one or more of the circuits 218-224 and any other circuits (not shown) that require power from the battery circuit 226. It should be appreciated that the battery circuit 226 may be implemented using any suitable implantable power source.

The signal processing circuit 220 is also electrically coupled to each electrode 702 and 704 (e.g., via the circuit 224) for sensing cardiac activity and/or stimulating cardiac tissue. Thus, in some cases, the electrodes 228 and 230 are used for stimulating cardiac tissue. In some cases, one or more of the electrodes 228 and 230 may be used for sensing cardiac activity (e.g., for near-field sensing and/or far-field sensing). For example, the electrodes 228 and 230 may correspond to the electrodes 126 and 128 of FIG. 1 (or some other combination of the electrodes of FIG. 1).

The sensing/pacing circuit 224 is electrically coupled to the electrodes 228 and 230 to receive electrical signals indicative of cardiac activity and/or to output cardiac stimulation signals (e.g., pacing pulses). To facilitate interfacing with these components, the sensing/pacing circuit 224 may comprise one or more of: a sensing circuit, an amplifier, a filter, a signal generator, a signal driver, a switching circuit, or other suitable circuits. Thus, the sensing/pacing circuit 224 may filter, amplify, and detect signals received from the electrodes 228 and 230. In addition, the sensing/pacing circuit 224 may generate, filter, and amplify signals sent to the electrodes 228 and 230.

The signal processing circuit 220 may process cardiac signals received via the sensing/pacing circuit 224 to identify cardiac events. For example, a microprocessor of the signal processing circuit 220 may be configured to acquire intra-cardiac electrogram data (and/or other cardiac related signal data) and identify P waves, R waves, T waves and other cardiac events of interest. Based on analysis of these cardiac events, the processing circuit may selectively generate stimulation signals (e.g., pacing pulses) to be delivered to cardiac tissue via one or more electrodes.

The signal processing circuit 220 also may control stimulation operations by controlling the signals generated by the sensing/pacing circuit 224. For example, a microprocessor of the signal processing circuit 220 may be configured to trigger the generation of pacing signals, specify pacing signal characteristics (e.g., energy level and duration), and inhibit pacing signals.

It should be appreciated that the signal processing circuit 220 may take various forms in different embodiments. For example, in some implementations, a single circuit (e.g., a microprocessor) may be employed to handle processing for both pressure sensing and cardiac operations. In other implementations, however, different circuits may be employed to provide the processing for these different operations.

Furthermore, in some embodiments, the frequency of the LC-resonant circuit 206 may be read by an external device. In such a case, the leadless intra-cardiac medical device 202 need not employ the circuit 218 or the capability of generating data representative of cardiac pressure. Rather, based on the frequency readings made by the external device, the external device will determine the cardiac pressure.

FIG. 3 is a simplified schematic and block diagram of an embodiment of a leadless intra-cardiac medical device 302 that comprises an active L-C resonant circuit pressure sensor.

Similar to the device 202 of FIG. 2, the device 302 comprises a housing 304, an L-C resonant circuit 306 and a circuit 308 that performs certain pressure sensing-related operations as well as cardiac sensing and/or pacing operations. The L-C resonant circuit 306 comprises a capacitive circuit 310 and an inductive circuit 312. A circuit 318 is configured to sense an oscillating signal 316 of the L-C resonant circuit 306. The circuit 308 comprises a signal processing circuit 320, a memory circuit 322, a sensing/pacing circuit 324, a battery circuit 326, and electrodes 328 and 330. Different combinations of these components may be employed in other embodiments constructed in accordance with the teachings herein.

In this embodiment, the L-C resonant circuit 306 is excited by an internal excitation circuit 314 instead of by external excitation signals. The excitation circuit 314 generates a signal (e.g., a single pulse, a set of pulses, or a periodic pulse signal) that serves to excite the L-C resonant circuit 306 and, if applicable, maintain oscillations in the L-C resonant circuit 306. To this end, the excitation circuit 314 may include an oscillator 332 or some other suitable signal generator circuit.

In some implementations, the signal processing circuit 320 (or some other suitable circuit of the device 302) controls the operation of the excitation circuit 314. For example, upon receipt of a suitable command from an external device (e.g., an external monitoring device) at the signal processing circuit 320, the excitation circuit 314 may be controlled to commence excitation of the L-C resonant circuit 306. Alternatively, the signal processing circuit 320 may be configured to initiate excitation at certain times (e.g., periodically). Concurrent with either of the above operations, the signal processing circuit 320 may commence processing of the received oscillating signal 316 and generating data representative of the pressure external to the device 302 based on this signal.

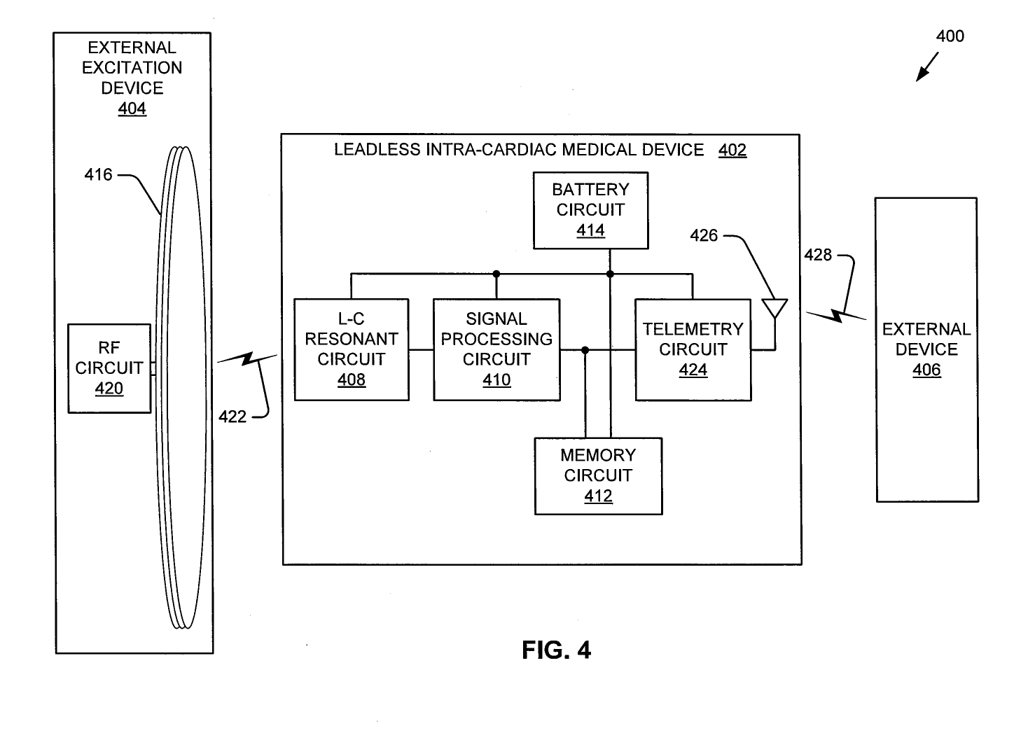

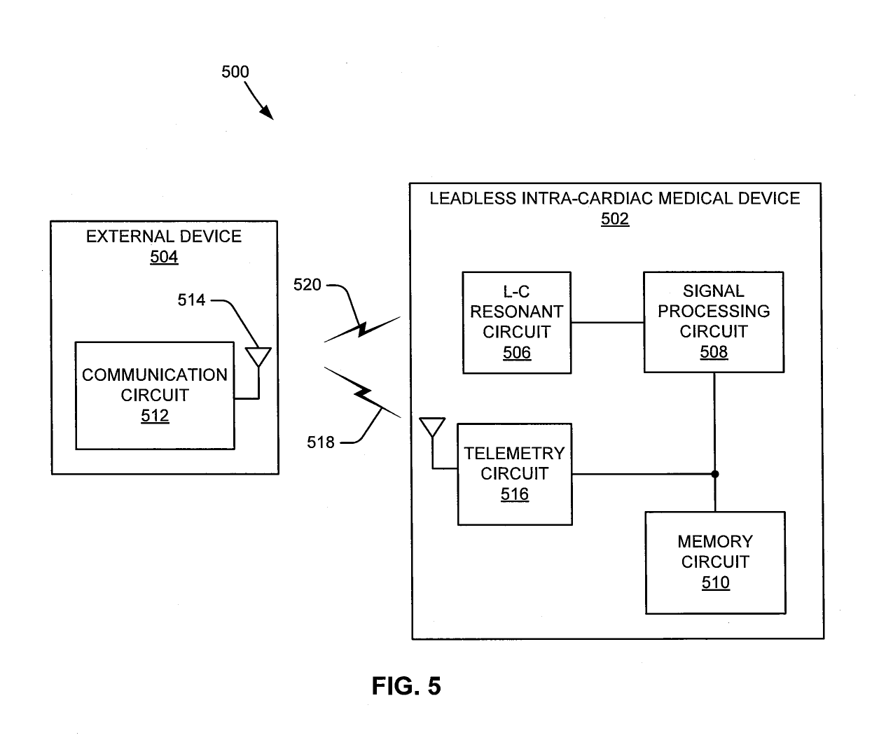

A leadless intra-cardiac medical device may communicate with external devices in different ways in different embodiments. FIGS. 4 and 5 depict two examples illustrating how a leadless intra-cardiac medical device may communicate with different types of external devices.

FIG. 4 illustrates an embodiment of a system 400 where a leadless intra-cardiac medical device 402 that is implanted in a patient (not shown) communicates with an external device 404 and an external device 406. In this example, the passive L-C resonant circuit is excited by RF signals 422 generated by the external device 404, and resulting oscillating signals in the excited L-C resonant circuit may be received by the external device 404. In addition, the device 402 communicates with the external device 406 (e.g., programmer, a home monitor, etc.) to, for example, upload and download information.

Similar to the device 202 of FIG. 2, the device 402 includes an L-C resonant circuit 408, a signal processing circuit 410, a memory circuit 412, and a battery circuit 414 that are electrically coupled with one another, if applicable. Several other circuits that would be included in the device 402 are not shown to reduce the complexity of FIG. 4.

The external device 404 includes an antenna 416 (e.g., a coil) that may be much larger than an effective antenna (e.g., the coil of the inductive circuit) for the L-C resonant circuit 408. For example, the antenna 416 may have dimensions of 12-20 centimeters in diameter while the coil of the inductive circuit may have dimensions of 3-4 millimeters in diameter. In this way, an RF circuit 420 of the external device 404 is able to more effectively couple relatively high frequency RF signals 422 through the tissue of a patient (not shown) to excite the L-C resonant circuit 408. The frequency of RF signals 422 may be at or near the resonant frequency of the L-C resonant circuit 408.

The device 402 also includes a telemetry circuit 424 and associated antenna 426 for communicating with the external device 406 via RF signals 428. For example, the external device 406 may communicate with the device 402 to initiate pressure sensing operations, to upload data generated by the pressure sensing operations, to control cardiac-related operations, and so on. Of note, the external device 406 may employ a smaller antenna (not shown) than the antenna 416 since less RF energy may be required to communicate with the device 402 than is required to excite the L-C resonant circuit 408 due to the use of lower frequency RF signals for this communication.

FIG. 5 illustrates an embodiment of a system 500 where a leadless intra-cardiac medical device 502 that is implanted in a patient (not shown) communicates with an external device 504. Similar to the device 402 of FIG. 4, the device 502 communicates with the external device 504 (e.g., programmer, a home monitor, etc.) to, for example, upload and download information. In addition, the device 502 includes an L-C resonant circuit 506, a signal processing circuit 508, and a memory circuit 510 that are electrically coupled in a suitable manner. Several other circuits that would be included in the device 502 are not shown to reduce the complexity of FIG. 5.

The configuration of FIG. 5 may be employed in cases where the external device 504 also includes the capability to excite a passive L-C resonant circuit pressure sensor of the device 502. For example, the external device 504 may include a communication circuit 512 that communicates via at least one antenna 514 with a telemetry circuit 516 of the device 502 (as represented by RF signals 518) and that excites the L-C resonant circuit 506 via RF signals 520.

The use of the single external device 504 for both operations is enabled based on the teachings herein because relative large reactive components may be employed for the L-C resonant circuit 506. For example, by using a sufficiently large leadless intra-cardiac medical device (e.g., in contrast with a relatively thin implantable lead), larger reactive components may be employed in the L-C resonant circuit 506. As a result, the L-C resonant circuit 506 may be implemented at a lower resonant frequency. Consequently, since a lower frequency RF signal is required in this case, the external device 504 may employ a smaller antenna (e.g., an antenna 514), yet still couple sufficient energy to the device 502 to excite the L-C resonant circuit 506.

The configuration of FIG. 5 also may be employed in cases where the device 502 employs an active L-C resonant circuit pressure sensor. In such a case, the communication circuit 512 would not transmit the RF signals 520 to excite the L-C resonant circuit 506. Rather, the communication circuit 512 would simply communicate with the telemetry circuit 516 via RF signals 518.

In view of the above, a leadless intra-cardiac medical device constructed in accordance with the teachings herein may provide one or more advantages over conventional medical devices. For example, such a device may provide sensing and/or pacing along with pressure sensing in a single implantable device. Moreover, implantation of the device (e.g., during and after the implant procedure) does not suffer from lead complications that may arise with a lead-based pressure sensor. The use of a leadless intra-cardiac medical device facilitates using larger pressure sensor components (e.g., capacitor and inductor), thereby enabling the use of a lower resonant frequency which may, in turn, enable the use of a smaller antenna coil at an external device. The use of a leadless intra-cardiac medical device enables power (from a battery circuit) to be readily provided for the pressure sensor, provides more effective telemetry for upload and downloading information (e.g., via on-board RF components), and facilitates acquisition of data over a period of time (e.g., via an on-board memory circuit).

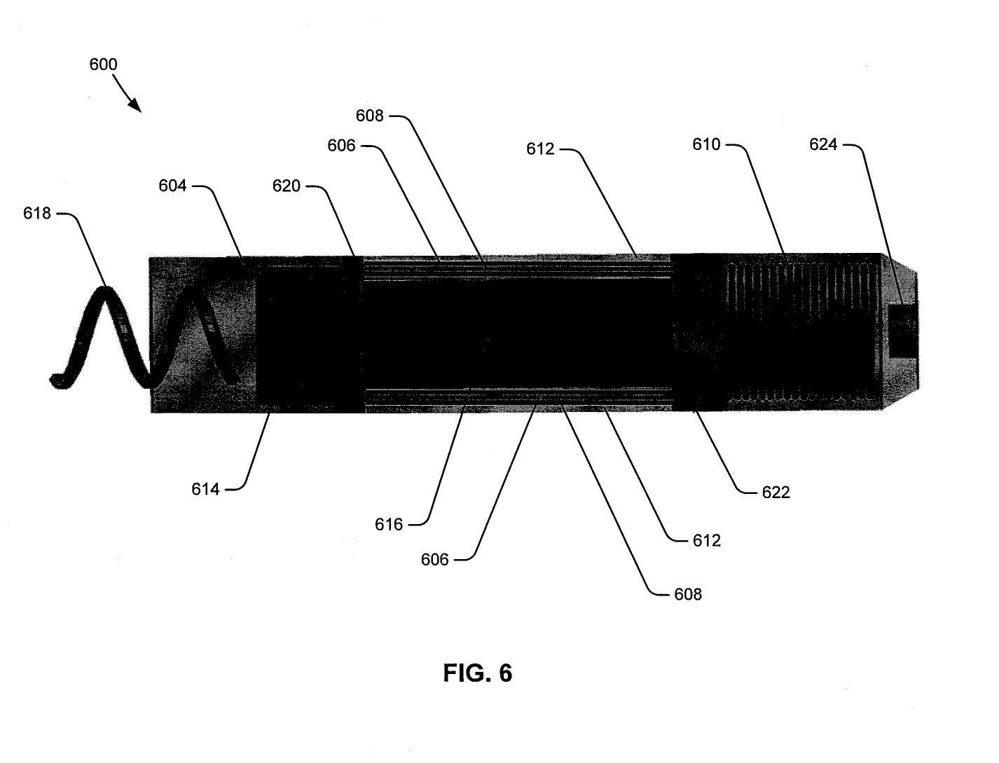

FIG. 6 illustrates several structural aspects of a sample embodiment of leadless intra-cardiac medical device. Specifically, a leadless intra-cardiac medical device 602 is depicted in a simplified sectional side view to show several interior components. The device 602 includes a housing 604 that houses LC-resonant circuit pressure sensing components and other cardiac-related components (e.g., for sensing and/or pacing).

As discussed herein, an LC-resonant circuit includes a capacitive circuit (comprising plates 606 and a dielectric material 608) and an inductive circuit (comprising a multi-layer coil 610).

The plates 606 of the capacitive circuit take the form of a cylinder or a partial cylinder. Here, each cylinder is oriented in a longitudinal direction along the longitudinal axis of the housing 604. That is, the longitudinal axis of each cylinder is parallel with (or, in some cases, the same as) longitudinal axis of the housing 604. Due to the large plate surface area that this configuration provides, in some embodiments, the plates 606 may be susceptible to relative deformation when the device 602 is subjected to changes in external pressure. Consequently, the L-C resonant circuit comprised of the capacitive circuit and the inductive circuit may be more sensitive to pressure changes, thereby facilitating more accurate pressure readings in some cases.

In some embodiments, the dielectric material 608 disposed between the plates 606 may be a relatively flexible material (e.g., a fiberglass material). In this way, external pressure induced on the device 602 may more easily cause the distance between the plates 606 to change. Thus, the resonant circuit comprised of the capacitive circuit and the inductive circuit will be more sensitive to pressure changes, thereby facilitating more accurate pressure readings in some cases.

A relatively flexible material 612 (e.g., a silicone-based material) may be disposed adjacent (e.g., next to or under) an exterior surface of the housing 604 and engaged with (e.g., disposed against, in contact with, etc.) the capacitive circuit (e.g., engaged with the plates 606). The flexible material 612 may thus serve to couple pressure waves to the capacitive circuit in an efficient manner. As discussed above, in some embodiments, the flexible material 612 may comprise a portion of the outer surface of the housing 604. In this case, the flexible material 612 itself will form part of the hermetic seal for the device 602, along with hermetic sealing (e.g., via adhesive or welding) between the flexible material 612 and housing 604. For example, a thin layer of fiberglass or sapphire (or some other suitable material) may be provided over an outer enclosure of the capacitive circuit (or directly over an outer plate 606 of the capacitive circuit).

In other embodiments (not shown in FIG. 6), the capacitive circuit may be housed entirely within (but located adjacent to) the housing 604. In such a case, the biocompatible housing 604 may provide the entire hermitic seal. In addition, the housing 604 will be sufficiently flexible (e.g., at least adjacent to the capacitive circuit) to couple pressure waves to the capacitive circuit. For example, the housing 604 may comprise a relatively thin outer layer (e.g., constructed of silicone, fiberglass, sapphire, or some other suitable material) that covers an outer enclosure of the capacitive circuit (or covers an outer plate 606 of the capacitive circuit). Alternatively, the entire housing 604 may be constructed of a flexible material.

As shown in FIG. 6, the inductive circuit may take the form of a cylindrical coil or some other coil-like structure. The inductive circuit may be constructed in various ways. In some embodiments, the inductive circuit is constructed with DFT wire (41% AG or less) or copper wire. The wire may be coated with, for example, ETFE or some other insulation material. In some embodiments, the wire may be relatively thin (e.g., 100 micrometers to 2 mils) so that the coil may have large number of turns, thereby providing a higher value of inductance for a given size coil.

The physical properties of the inductive circuit (e.g., the number of coil turns) and the capacitive circuit (e.g., size and distance between the plates 606) are selected to provide a desired resonant frequency for the L-C resonant circuit. In some embodiments, the resonant circuit has a resonant frequency of 35 MHz or less (e.g., 30 MHz). Such a circuit may be compatible with other types of passive pressure sensors.

In some embodiments, the resonant circuit has a resonant frequency of 10 MHz or less. This lower resonant frequency may be achieved, for example, through the use of larger (e.g., in size and shape) components in the L-C resonant circuit by employing an implantable device of sufficient size in accordance with the teachings herein. Such a circuit may advantageously enable the use of a smaller transmission coil at the external monitoring system or other similar device (e.g., for inducing an RF excitation signal at a passive L-C resonant circuit pressure sensor). Consequently, a more portable external monitoring system (or other device) may be employed to acquire pressure readings from a passive pressure sensor constructed in accordance with the teachings herein. Alternative, this smaller size may enable the transmission coil for RF excitation to be incorporated into a single external device (e.g., a programmer) used for communicating with an implanted leadless intra-cardiac medical device (e.g., a pacemaker, an ICD, etc.).

The device 604 also includes a circuit 614 (e.g., comprising an integrated circuit and/or discrete circuits) for performing pressure sensing-related operations as taught herein. The circuit 614 is powered by a battery circuit 616.

The device 604 also includes components for performing other cardiac-related operations. In this example, the device 602 includes electrodes 618, 620, 622, and 624.

The circuit 614 also may include circuitry for acquiring and processing signals indicative of cardiac activity and for applying stimulation signals to cardiac tissue. For example, for sensing operations, at least one sensing circuit is coupled to one or more of the electrodes 618-624 for measuring cardiac electrical activity. In addition, for stimulation operations, at least one signal generator circuit is coupled to one or more of the electrodes 618-624 for stimulating cardiac tissue.

Electrodes of the device 602 may be configured in different ways for different stimulation operations. In some implementations, the electrode 618 acts as a cathode and the electrode 620 acts as an anode. In other implementations, the electrode 618 acts as the cathode and the housing 604 (e.g., comprising a conductive biocompatible material) acts as the anode.

Electrodes of the device 602 may be configured in different ways for different cardiac sensing operations. For example, in some implementations, the electrodes 618 and 620 are used for acquiring near-field signals, while the electrodes 622 and 624 are used for acquiring far-field signals. For sensing, the housing 604 (e.g., comprising a conductive material) and/or another electrode may act as a reference electrode (e.g., ground). As used herein, the term near-field signal refers to a signal that originates in a local chamber (i.e., the same chamber) where the corresponding sense electrodes are located. Conversely, the term far-field signal refers to a signal that originates in a chamber other than the local chamber where the corresponding sense electrodes are located.

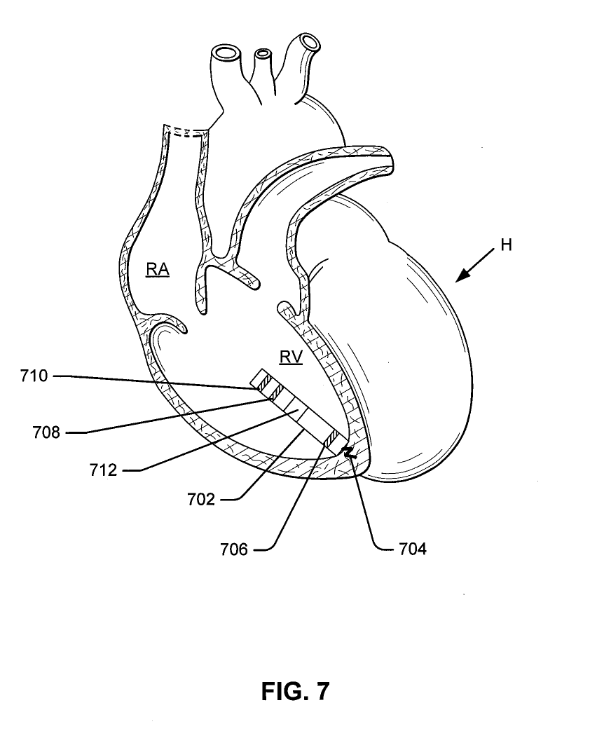

Here, depending on the ratio of electrode surface areas, spacing between electrodes, and tissue contact, a pair of electrodes may be employed to effectively sense both near-field and far-field electrical activity. In some implementations (e.g., as depicted in FIG. 7), a leadless intra-cardiac medical device may be implanted with an electrode used as the cathode in pacing attached endocardially to the myocardium of the heart and an electrode used as the anode protruding into a chamber of the heart. In some implementations, a leadless intra-cardiac medical device may be implanted with the cathode attached at the epicardial surface of the heart and the anode on the other external face of the device. Because of the larger surface area of the anode and its contact to low-impedance fluid, the sensed electrical activity will include a significant far-field component that has less high-frequency content (in addition to the near-field signal of electrical activity near the cathode).

The electrodes 620-624 may be implemented as forming part of the housing 604 or may be implemented upon (i.e., around) a recessed section of the housing 604. In the latter case, if the housing 604 is conductive, the electrodes 620-624 may lie on top of an insulator (not shown) that separates the bottom surface of these electrodes from an upper surface of the housing 604.

To facilitate long-term implant within a patient, all external surfaces and materials of the leadless intra-cardiac medical device 602 comprise biocompatible materials. For example, the housing 604 may be constructed of titanium, a ceramic material, or some other suitable biocompatible material. The electrodes 618-624 may be constructed of titanium or some other suitable conductive and biocompatible material. In addition, the flexible material 612 may be constructed of polyurethane, silicone or some other suitable flexible and biocompatible (and, optionally electrically insulating) material. Furthermore, in embodiments that employ insulators, the insulators may be constructed of ceramic, polyurethane, silicone or some other suitable electrically insulating and biocompatible material.

To facilitate long-term implant, the leadless intra-cardiac medical device 602 is hermetically sealed. To this end, hermetic sealing techniques may be employs to attach the flexible material 612 to the housing 604. In addition, hermetically sealed feedthroughs may be employed in some embodiments to electrically couple the electrodes 618-624 to internal conductors of the leadless intra-cardiac medical device 602. Alternatively, feedthroughs may not be employed in embodiments where the electrodes 618-624 are part of a hermetic housing 604. In such a case, an electrical connection may be made to an interior surface of these electrodes.

In some embodiments, the leadless intra-cardiac medical device 602 is sized to facilitate venous-based implant to a single cardiac chamber (e.g., the RV). For example, the housing 604 may have a cross-sectional width (e.g., outer diameter) of 12 french or less in some embodiments. In addition, to accommodate the internal circuitry (in particular, the battery of the battery circuit 616), the housing 604 may have a length of at least 30 millimeters in some embodiments. It should be appreciated, however, that different dimension may be employed in other embodiments. For example, the outer diameter of the housing 604 may be 7 or 8 french of less in some embodiments. Also, the length of the housing 604 may be 30 millimeters or less in some embodiments.

In some embodiments it is desirable to place the proximal electrodes as close to an adjacent chamber as possible (e.g., to facilitate far-field sensing). In such a case, the housing 604 may have a length of 60 millimeters or more.

FIG. 7 illustrates an example of how a leadless intra-cardiac medical device 702 may be implanted in a chamber of a heart H. In this example, the leadless intra-cardiac medical device 702 is implanted at the apex of the right ventricle (RV) of the heart H. In accordance with the teachings herein, the device 702 includes an L-C resonant circuit pressure sensor 712 for measure RV pressure.

A distal section of the leadless intra-cardiac medical device 702 comprises a helix electrode 704 that is actively affixed to an inner wall of the RV. The helix electrode 704 in combination with a ring electrode 706 may be used for near-field sensing of RV events. In addition, bipolar electrodes 708 and 710 at a proximal section of the leadless intra-cardiac medical device 702 may be employed for far-field sensing of RA events and/or other cardiac events. Here, the electrodes 708 and 710 may be optimized for such far-field sensing based on, for example, one or more of: placement of the electrodes 708 and 710 at a proximal section of the leadless intra-cardiac medical device 702, increased spacing between the electrodes 708 and 710, or increased sizing of the electrodes 708 and 710.

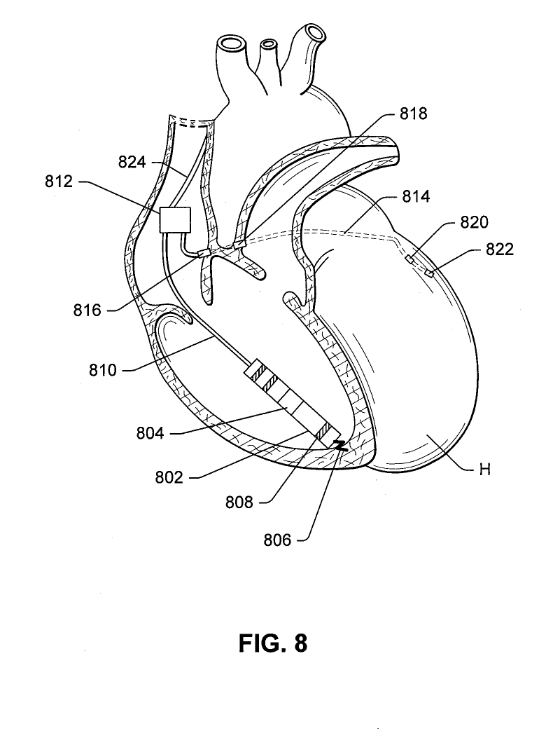

FIG. 8 illustrates an embodiment where a leadless intra-cardiac medical device 802 is connected to other electrodes (but not intravenous leads). In accordance with the teachings herein, the device 802 includes an L-C resonant circuit pressure sensor 804 for measure RV pressure. In addition, in this example, a distal section of the leadless intra-cardiac medical device 802 comprises a helix electrode 806 that is actively affixed to an inner wall of the RV and a ring electrode 808.

The leadless intra-cardiac medical device 802 is connected via at least one conductor 810 and a junction box 812 to at least one other conductor 814 that is coupled to several electrodes 816-822. In this example, the junction box 812 is held in place upon implant through the use of at least one mechanical support (e.g., attachment) structure 824.

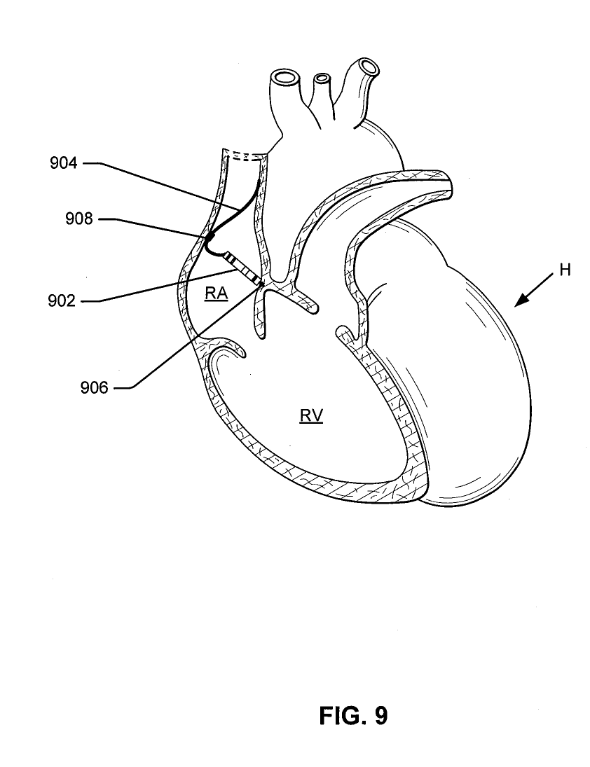

FIG. 9 illustrates another embodiment where a leadless intra-cardiac medical device 902 constructed in accordance with the teachings herein comprises at least one mechanical support (e.g., attachment) structure 904. The mechanical support structure 904 assists in holding the leadless intra-cardiac medical device 904 in place within the heart H upon implant. For example, the mechanical support structure 904 may expand upon implant (e.g., after removal of an implant sleeve) and exert forces on opposite walls of the right atrium (RA) as shown, thereby helping to hold the leadless intra-cardiac medical device 902 in place within the chamber. Thus, several sections of the structure 904 may be actively attached to the inner wall W (e.g., via a mechanical or chemical-based attachment technique) and/or eventually become passively attached to the inner wall W (e.g., by buildup of intima over the section). Thus, the leadless intra-cardiac medical device 902 may be held firmly in place by action of the mechanical support structure 904 and by action of a helix structure 906 at the distal section of the leadless intra-cardiac medical device 902 (e.g., implanted between the tricuspid valve and the coronary sinus ostium for sensing/pacing the RV).

FIG. 9 also illustrates that a mechanical support structure may comprise one or more electrodes. In this example, an electrode 908 is positioned along the mechanical support structure 904 such that the electrode 908 is in contact with a wall of the RA to facilitate sensing and/or pacing in the RA. The electrode 908 is electrically coupled to the internal circuitry (not shown) of the leadless intra-cardiac medical device 902 via conductors (not shown) that are incorporated into the mechanical support structure 904.

A mechanical support structure as taught herein may take different forms in different implementations. For example, as discussed above, a mechanical support structure (e.g., comprising a single attachment member or multiple attachment members working in cooperation) may be predisposed to rest against multiple inner walls of a heart. As another example, a mechanical support structure may be configured to facilitate passive or active attachment to an inner wall. A mechanical support structure may be composed of Nitinol, polyurethane, or some other suitable biocompatible material.

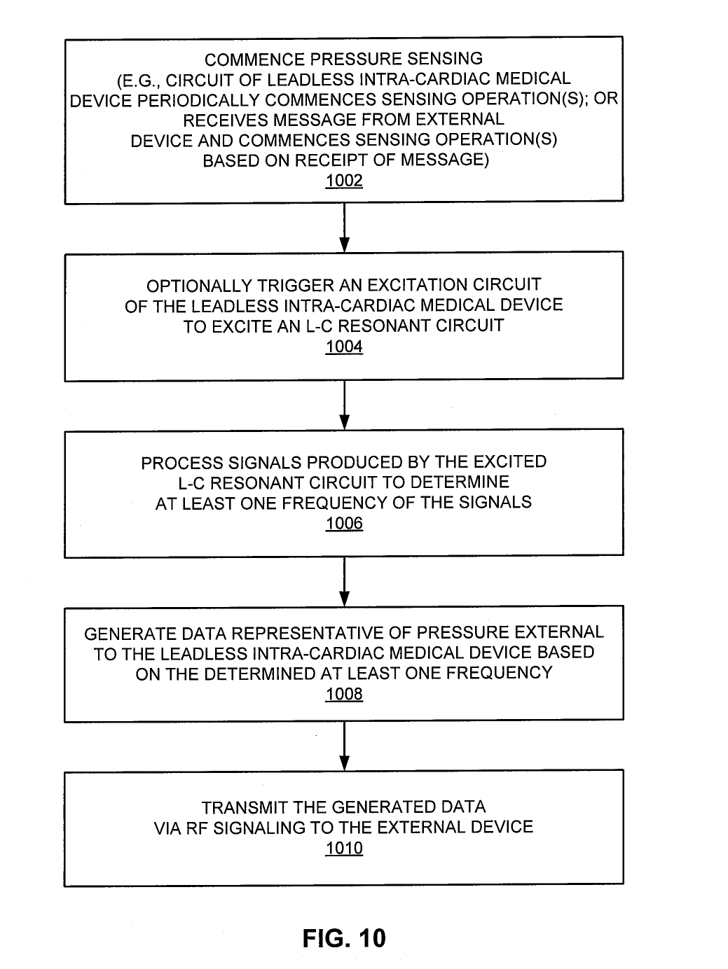

Representative operations relating to pressure sensing by an embodiment of a leadless intra-cardiac medical device in accordance with the teachings herein will be described in more detail in conjunction with the flowchart of FIG. 10. For convenience, the operations of FIG. 10 (or any other operations discussed or taught herein) may be described as being performed by specific components. It should be appreciated, however, that these operations may be performed by other types of components and may be performed using a different number of components. It also should be appreciated that one or more of the operations described herein may not be employed in a given implementation.

As represented by block 1002 of FIG. 10, at some point in time, the leadless intra-cardiac medical device commences pressure sensing. For example, the device may receive a message (e.g., a command from an external monitor device) or some other type of signal (e.g., an RF signal from an external device that provides an RF excitation signal) from an external device. As a result of the receipt of the message or signal, the leadless intra-cardiac medical device (e.g., circuit 120, 208, 308, or 614, etc.) may commence processing the oscillating signal from the L-C resonant circuit and generating data representative of pressure waves. As another example, the leadless intra-cardiac medical device may be configured to periodically conduct these pressure sensing operations.

As represented by block 1004, in embodiments where the leadless intra-cardiac medical device comprises an active L-C resonant circuit, the device may trigger an excitation circuit to excite the L-C resonant circuit. As discussed herein, this trigger may be based on receipt of a message or signal, based on a pressure sensing schedule (e.g., periodic sensing) implemented at the device, or based on some other factor(s).

As represented by block 1006, the leadless intra-cardiac medical device processes signal produced by the excited L-C resonant circuit to determine at least one frequency of the signals. For example, the device may monitor the frequency of the signals over a period of time to determine how the frequency varies over that period of time.

As represented by block 1008, the leadless intra-cardiac medical device generates data representative of pressure external to the device based on the at least one frequency determined at block 1006. For example, the device may generate data indicative of how the measured cardiac pressure varies over a designated period of time.

As represented by block 1010, the leadless intra-cardiac medical device transmits the data generated at block 1008 to an external device (e.g., an external monitoring device) via RF signaling. For example, the leadless intra-cardiac medical device may send this information on-demand (e.g., in response to a message), according to a schedule (e.g., periodically), or in some other suitable manner.

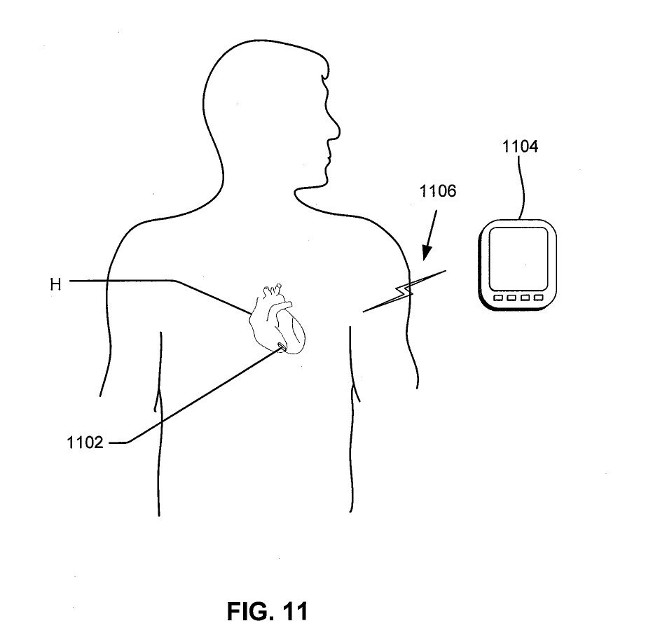

FIG. 11 is a simplified diagram of a device 1102 (implanted within a patient P) that communicates with a device 1104 that is located external to the patient P. The implanted device 1102 and the external device 1104 communicate with one another via a wireless communication link 1106 (as represented by the depicted wireless symbol).

In the illustrated example, the implanted device 1102 is a leadless intra-cardiac medical device including an L-C resonant circuit pressure sensor (not shown) in accordance with the teachings herein. For example, the implanted device 1102 may be a pacemaker, an implantable cardioverter defibrillator, or some other similar device. It should be appreciated, however, that the implanted device 1102 may take other forms.

The external device 1104 also may take various forms. For example, the external device 1104 may be a base station, a programmer, a home safety monitor, a personal monitor, a follow-up monitor, a wearable monitor, or some other type of device that is configured to communicate with the implanted device 1102.

The communication link 1106 may be used to transfer information between the devices 1102 and 1104 in conjunction with various applications such as remote home-monitoring, clinical visits, data acquisition, remote follow-up, and portable or wearable patient monitoring/control systems. For example, when information needs to be transferred between the devices 1102 and 1104, the patient P moves into a position that is relatively close to the external device 1104, or vice versa.

The external device 1104 may send information it receives from an implanted device to another device (e.g., that may provide a more convenient means for a physician to review the information). For example, the external device 1104 may send the information to a web server (not shown). In this way, the physician may remotely access the information (e.g., by accessing a website). The physician may then review the information uploaded from the implantable device to determine whether medical intervention is warranted.

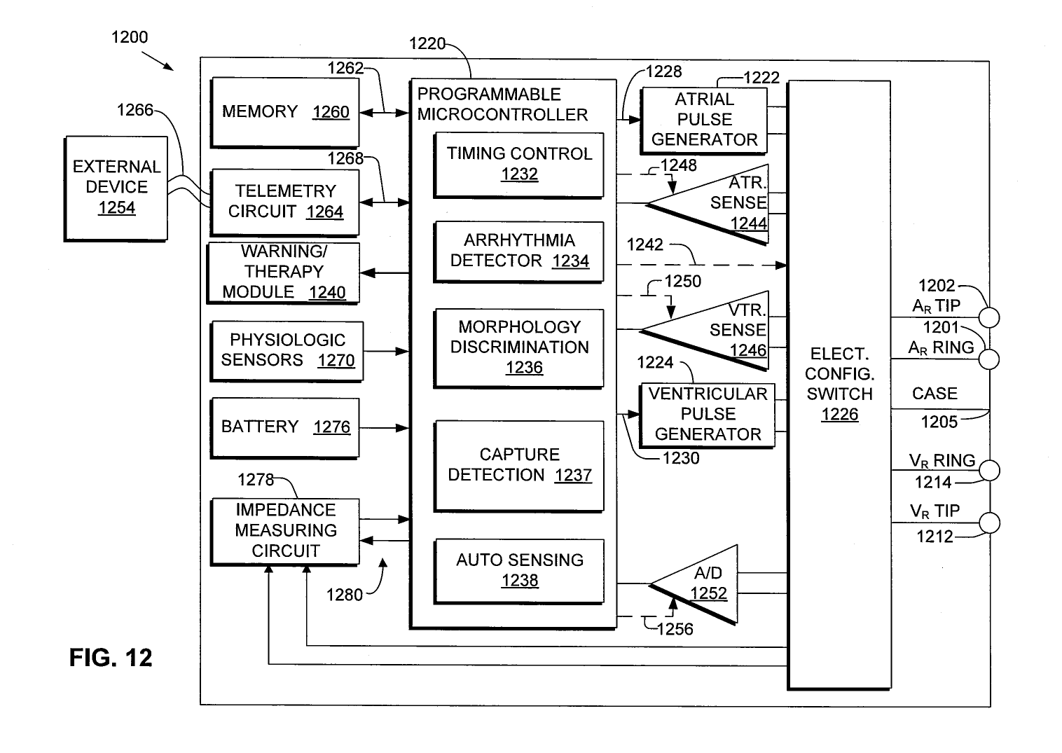

FIG. 12 illustrates sample components of an embodiment of an implantable leadless intra-cardiac medical device 1200 (e.g., a stimulation device such as an implantable cardioverter defibrillator, a pacemaker, etc., or a monitoring device) that may be configured in accordance with the various embodiments that are described herein. It is to be appreciated and understood that other cardiac devices can be used and that the description below is given, in its specific context, to assist the reader in understanding, with more clarity, the embodiments described herein.

In various embodiments, the device 1200 may be adapted to treat both fast and slow arrhythmias with stimulation therapy, including cardioversion, defibrillation, and pacing stimulation. While a particular multi-chamber device is shown, it is to be appreciated and understood that this is done for illustration purposes. Thus, the techniques and methods described below can be implemented in connection with any suitably configured or configurable device. Accordingly, one of skill in the art could readily duplicate, eliminate, or disable the appropriate circuitry in any desired combination to provide a device capable of treating the appropriate chamber(s) with, for example, cardioversion, defibrillation, and pacing stimulation.

A housing 1205 for the device 1200 is often referred to as the “can”, “case” or “case electrode”, and may be programmably selected to act as the return electrode for all “unipolar” modes. The housing 1205 may further be used as a return electrode alone or in combination with one or more coil electrodes (not shown) for shocking purposes. As discussed herein, the housing 1205 may be constructed of a biocompatible material (e.g., titanium) to facilitate implant within a patient.

The device 1200 further includes a plurality of terminals that connect the internal circuitry of the device 1200 to electrodes 1201, 1202, 1212, and 1214 of the device 1200. Here, the name of the electrodes to which each terminal is connected is shown next to that terminal. The device 1200 may be configured to include various other terminals depending on the requirements of a given application. Thus, it should be appreciated that other terminals (and associated circuitry) may be employed in other embodiments.

To achieve right atrial sensing and pacing, a right atrial tip terminal (A_{R} TIP) is adapted for connection to a right atrial tip electrode 1202. A right atrial ring terminal (A_{R} RING) may also be included and adapted for connection to a right atrial ring electrode 1201. To achieve right ventricular sensing and pacing, a right ventricular tip terminal (V_{R} TIP) and a right ventricular ring terminal (V_{R} RING) are adapted for connection to a right ventricular tip electrode 1212 and a right ventricular ring electrode 1214, respectively.

At the core of the device 1200 is a programmable microcontroller 1220 that controls the various modes of stimulation therapy. As is well known in the art, microcontroller 1220 typically includes a microprocessor, or equivalent control circuitry, designed specifically for controlling the delivery of stimulation therapy, and may further include memory such as RAM, ROM and flash memory, logic and timing circuitry, state machine circuitry, and I/O circuitry. Typically, microcontroller 1220 includes the ability to process or monitor input signals (data or information) as controlled by a program code stored in a designated block of memory. The type of microcontroller is not critical to the described implementations. Rather, any suitable microcontroller 1220 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. No. 4,712,555 (Thornander et al.) and U.S. Pat. No. 4,944,298 (Sholder), all of which are incorporated by reference herein. For a more detailed description of the various timing intervals that may be used within the device and their inter-relationship, see U.S. Pat. No. 4,788,980 (Mann et al.), also incorporated herein by reference.

FIG. 12 also shows an atrial pulse generator 1222 and a ventricular pulse generator 1224 that generate pacing stimulation pulses for delivery by the right atrial electrodes, the right ventricular electrode, or some combination of these electrodes via an electrode configuration switch 1226. It is understood that in order to provide stimulation therapy in each of the four chambers of the heart, the atrial and ventricular pulse generators 1222 and 1224 may include dedicated, independent pulse generators, multiplexed pulse generators, or shared pulse generators. The pulse generators 1222 and 1224 are controlled by the microcontroller 1220 via appropriate control signals 1228 and 1230, respectively, to trigger or inhibit the stimulation pulses.

Microcontroller 1220 further includes timing control circuitry 1232 to control the timing of the stimulation pulses (e.g., pacing rate, atrioventricular (AV) delay, inter-atrial conduction (A-A) delay, or inter-ventricular conduction (V-V) delay, etc.) or other operations, as well as to keep track of the timing of refractory periods, blanking intervals, noise detection windows, evoked response windows, alert intervals, marker channel timing, etc., as known in the art.

Microcontroller 1220 further includes an arrhythmia detector 1234. The arrhythmia detector 1234 may be utilized by the device 1200 for determining desirable times to administer various therapies. The arrhythmia detector 1234 may be implemented, for example, in hardware as part of the microcontroller 1220, or as software/firmware instructions programmed into the device 1200 and executed on the microcontroller 1220 during certain modes of operation.

Microcontroller 1220 may include a morphology discrimination module 1236, a capture detection module 1237 and an auto sensing module 1238. These modules are optionally used to implement various exemplary recognition algorithms or methods. The aforementioned components may be implemented, for example, in hardware as part of the microcontroller 1220, or as software/firmware instructions programmed into the device 1200 and executed on the microcontroller 1220 during certain modes of operation.

The electrode configuration switch 1226 includes a plurality of switches for connecting the desired terminals (e.g., that are connected to electrodes, coils, sensors, etc.) to the appropriate I/O circuits, thereby providing complete terminal and, hence, electrode programmability. Accordingly, switch 1226, in response to a control signal 1242 from the microcontroller 1220, may be used to determine the polarity of the stimulation pulses (e.g., unipolar, bipolar, combipolar, etc.) by selectively closing the appropriate combination of switches (not shown) as is known in the art.

Atrial sensing circuits (ATR. SENSE) 1244 and ventricular sensing circuits (VTR. SENSE) 1246 may also be selectively coupled to the right atrial electrodes and the right ventricular electrodes through the switch 1226 for detecting the presence of cardiac activity in each of the four chambers of the heart. Accordingly, the atrial and ventricular sensing circuits 1244 and 1246 may include dedicated sense amplifiers, multiplexed amplifiers, or shared amplifiers. Switch 1226 determines the “sensing polarity” of the cardiac signal by selectively closing the appropriate switches, as is also known in the art. In this way, the clinician may program the sensing polarity independent of the stimulation polarity. The sensing circuits (e.g., circuits 1244 and 1246) are optionally capable of obtaining information indicative of tissue capture.

Each sensing circuit 1244 and 1246 preferably employs one or more low power, precision amplifiers with programmable gain, automatic gain control, bandpass filtering, a threshold detection circuit, or some combination of these components, to selectively sense the cardiac signal of interest. The automatic gain control enables the device 1200 to deal effectively with the difficult problem of sensing the low amplitude signal characteristics of atrial or ventricular fibrillation.

The outputs of the atrial and ventricular sensing circuits 1244 and 1246 are connected to the microcontroller 1220, which, in turn, is able to trigger or inhibit the atrial and ventricular pulse generators 1222 and 1224, respectively, in a demand fashion in response to the absence or presence of cardiac activity in the appropriate chambers of the heart. Furthermore, as described herein, the microcontroller 1220 is also capable of analyzing information output from the sensing circuits 1244 and 1246, a data acquisition system 1252, or both. This information may be used to determine or detect whether and to what degree tissue capture has occurred and to program a pulse, or pulses, in response to such determinations. The sensing circuits 1244 and 1246, in turn, receive control signals over signal lines 1248 and 1250, respectively, from the microcontroller 1220 for purposes of controlling the gain, threshold, polarization charge removal circuitry (not shown), and the timing of any blocking circuitry (not shown) coupled to the inputs of the sensing circuits 1244 and 1246 as is known in the art.

For arrhythmia detection, the device 1200 utilizes the atrial and ventricular sensing circuits 1244 and 1246 to sense cardiac signals to determine whether a rhythm is physiologic or pathologic. It should be appreciated that other components may be used to detect arrhythmia depending on the system objectives. In reference to arrhythmias, as used herein, “sensing” is reserved for the noting of an electrical signal or obtaining data (information), and “detection” is the processing (analysis) of these sensed signals and noting the presence of an arrhythmia.

Timing intervals between sensed events (e.g., P-waves, R-waves, and depolarization signals associated with fibrillation) may be classified by the arrhythmia detector 1234 of the microcontroller 1220 by comparing them to a predefined rate zone limit (e.g., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”). Similar rules may be applied to the atrial channel to determine if there is an atrial tachyarrhythmia or atrial fibrillation with appropriate classification and intervention.

Cardiac signals or other signals may be applied to inputs of an analog-to-digital (A/D) data acquisition system 1252. The data acquisition system 1252 is configured (e.g., via signal line 1256) to acquire intra-cardiac electrogram (“IEGM”) signals or other signals, convert the raw analog data into a digital signal, and store the digital signals for later processing, for telemetric transmission to an external device 1254, or both. For example, the data acquisition system 1252 may be coupled to the right atrial electrodes and the right ventricular electrodes through the switch 1226 to sample cardiac signals across any pair of desired electrodes.

The data acquisition system 1252 also may be coupled to receive signals from other input devices. For example, the data acquisition system 1252 may sample signals from a physiologic sensor 1270 or other components shown in FIG. 12 (connections not shown).

The microcontroller 1220 is further coupled to a memory 1260 by a suitable data/address bus 1262, wherein the programmable operating parameters used by the microcontroller 1220 are stored and modified, as required, in order to customize the operation of the device 1200 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart H within each respective tier of therapy. One feature of the described embodiments is the ability to sense and store a relatively large amount of data (e.g., from the data acquisition system 1252), which data may then be used for subsequent analysis to guide the programming of the device 1200.

Advantageously, the operating parameters of the implantable device 1200 may be non-invasively programmed into the memory 1260 through a telemetry circuit 1264 in telemetric communication via communication link 1266 with the external device 1254, such as a programmer, transtelephonic transceiver, a diagnostic system analyzer or some other device. The microcontroller 1220 activates the telemetry circuit 1264 with a control signal (e.g., via bus 1268). The telemetry circuit 1264 advantageously allows intra-cardiac electrograms and status information relating to the operation of the device 1200 (as contained in the microcontroller 1220 or memory 1260) to be sent to the external device 1254 through an established communication link 1266.

The device 1200 includes one or more physiologic sensors 1270. At least one sensor 1270 comprises a pressure sensor as taught herein. In some embodiments, the device 1200 may include a “rate-responsive” sensor that may provide, for example, information to aid in adjustment of pacing stimulation rate according to the exercise state of the patient. One or more physiologic sensors 1270 (e.g., a pressure sensor) may be used to detect changes in cardiac output, changes in the physiological condition of the heart, or diurnal changes in activity (e.g., detecting sleep and wake states). Accordingly, the microcontroller 1220 may respond to this sensing by adjusting the various pacing parameters (such as rate, A-V Delay, V-V Delay, etc.) at which the atrial and ventricular pulse generators 1222 and 1224 generate stimulation pulses.

While shown as being included within the device 1200, it is to be understood that a physiologic sensor 1270 may also be external to the device 1200, yet still be implanted within or carried by the patient. Examples of physiologic sensors that may be implemented in conjunction with the device 1200 include sensors that sense respiration rate, pH of blood, ventricular gradient, oxygen saturation, blood pressure and so forth. Another sensor that may be used is one that detects activity variance, wherein an activity sensor is monitored diurnally to detect the low variance in the measurement corresponding to the sleep state. For a more detailed description of an activity variance sensor, the reader is directed to U.S. Pat. No. 5,476,483 (Bornzin et al.), which patent is hereby incorporated by reference.

The one or more physiologic sensors 1270 may optionally include one or more of components to help detect movement (via, e.g., a position sensor or an accelerometer) and minute ventilation (via an MV sensor) in the patient. Signals generated by the position sensor and MV sensor may be passed to the microcontroller 1220 for analysis in determining whether to adjust the pacing rate, etc. The microcontroller 1220 may thus monitor the signals for indications of the patient's position and activity status, such as whether the patient is climbing up stairs or descending down stairs or whether the patient is sitting up after lying down.

The device 1200 additionally includes a battery 1276 that provides operating power to all of the circuits shown in FIG. 12. For a device 1200 which employs shocking therapy, the battery 1276 is capable of operating at low current drains (e.g., preferably less than 10 μA) for long periods of time, and is capable of providing high-current pulses (for capacitor charging) when the patient requires a shock pulse (e.g., preferably, in excess of 2 A, at voltages above 200 V, for periods of 10 seconds or more). The battery 1276 also desirably has a predictable discharge characteristic so that elective replacement time can be detected. Accordingly, the device 1200 preferably employs lithium or other suitable battery technology.

The device 1200 can further include magnet detection circuitry (not shown), coupled to the microcontroller 1220, to detect when a magnet is placed over the device 1200. A magnet may be used by a clinician to perform various test functions of the device 1200 and to signal the microcontroller 1220 that the external device 1254 is in place to receive data from or transmit data to the microcontroller 1220 through the telemetry circuit 1264.

The device 1200 further includes an impedance measuring circuit 1278 that is enabled by the microcontroller 1220 via a control signal 1280. The known uses for an impedance measuring circuit 1278 include, but are not limited to, electrode impedance surveillance during the acute and chronic phases for proper performance, electrode positioning or dislodgement; detecting operable electrodes and automatically switching to an operable pair if dislodgement occurs; measuring respiration or minute ventilation; measuring thoracic impedance for determining shock thresholds; detecting when the device 1200 has been implanted; measuring stroke volume; and detecting the opening of heart valves, etc. The impedance measuring circuit 1278 is advantageously coupled to the switch 1226 so that any desired electrode may be used.

In the case where the device 1200 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 1220 may include a shocking circuit (not shown). The shocking circuit generates shocking pulses of low (e.g., up to 0.5 J), moderate (e.g., 0.5 J to 10 J), or high energy (e.g., 11 J to 40 J), as controlled by the microcontroller 1220. Such shocking pulses may be applied to the patient's heart H through, for example, two or more shocking electrodes (not shown).

Cardioversion level shocks are generally considered to be of low to moderate energy level (so as to minimize pain felt by the patient), be synchronized with an R-wave, pertain to the treatment of tachycardia, or some combination of the above. Defibrillation shocks are generally of moderate to high energy level (i.e., corresponding to thresholds in the range of 5 J to 40 J), delivered asynchronously (since R-waves may be too disorganized), and pertaining to the treatment of fibrillation. Accordingly, the microcontroller 1220 is capable of controlling the synchronous or asynchronous delivery of the shocking pulses.

The device 1200 thus illustrates several components that may provide the implantable intra-cardiac medical device functionality described above in conjunction with FIGS. 1-11. For example, the microcontroller 1220 (e.g., a processor providing signal processing functionality) may implement or support at least a portion of the processing functionality discussed above. Also, one or more of the switch 1226, the sense circuits 1244, 1246, and the data acquisition system 1252 may acquire cardiac signals that are used in the signal acquisition operations discussed above. Similarly, one or more of the switch 1226 and the pulse generator circuits 1222, 1224 may be used to provide stimulation signals that are used in the cardiac stimulation operations discussed above. The data described above (e.g., pressure data and/or cardiac data) may be stored in the data memory 1260. The physiologic sensors 1270 may comprise the pressure sensor(s) discussed above. Thus, in general, the processing circuitry described herein (e.g., the circuit 120, 208, 308, or 614, etc.) may correspond to one or more of the illustrated components of the device 1200.

It should be appreciated that various modifications may be incorporated into the disclosed embodiments based on the teachings herein. For example, the structure and functionality taught herein may be incorporated into types of devices other than the specific types of devices described above. In addition, pressure sensors for a leadless intra-cardiac medical device may be implemented in different ways in different embodiments based on the teachings herein. Different types of structural members and mechanical support structures may be employed in conjunction with a leadless intra-cardiac medical device as taught herein. Also, various algorithms or techniques may be employed to monitor pressure in various cardiac chambers (e.g., RA, RV, LA, and LV chambers) in accordance with the teachings herein. In some aspects, an apparatus or any component of an apparatus may be configured to provide functionality as taught herein by, for example, manufacturing (e.g., fabricating) the apparatus or component so that it will provide the functionality, by programming the apparatus or component so that it will provide the functionality, or through the use of some other suitable means.

It should be appreciated from the above that the various structures and functions described herein may be implemented in a variety of ways. Different embodiments of such an apparatus may include a variety of hardware and software processing components. In some embodiments, hardware components such as processors, controllers, state machines, logic, or some combination of these components, may be used to implement the described components or circuits.

In some embodiments, code including instructions (e.g., software, firmware, middleware, etc.) may be executed on one or more processing devices to implement one or more of the described functions or components. The code and associated components (e.g., data structures and other components used by the code or used to execute the code) may be stored in an appropriate data memory that is readable by a processing device (e.g., commonly referred to as a computer-readable medium).

Moreover, some of the operations described herein may be performed by a device that is located externally with respect to the body of the patient. For example, an implanted device may send raw data or processed data to an external device that then performs the necessary processing.

The components and functions described herein may be connected or coupled in many different ways. The manner in which this is done may depend, in part, on whether and how the components are separated from the other components. In some embodiments, some of the connections or couplings represented by the lead lines in the drawings may be in an integrated circuit, on a circuit board or implemented as discrete wires or in other ways.

The signals discussed herein may take various forms. For example, in some embodiments a signal may comprise electrical signals transmitted over a wire, light pulses transmitted through an optical medium such as an optical fiber or air, or RF waves transmitted through a medium such as air, and so on. In addition, a plurality of signals may be collectively referred to as a signal herein. The signals discussed above also may take the form of data. For example, in some embodiments an application program may send a signal to another application program. Such a signal may be stored in a data memory.

Moreover, the recited order of the blocks in the processes disclosed herein is simply an example of a suitable approach. Thus, operations associated with such blocks may be rearranged while remaining within the scope of the present disclosure. Similarly, the accompanying method claims present operations in a sample order, and are not necessarily limited to the specific order presented.

Also, it should be understood that any reference to elements herein using a designation such as “first,” “second,” and so forth does not generally limit the quantity or order of those elements. Rather, these designations may be used herein as a convenient method of distinguishing between two or more different elements or instances of an element. Thus, a reference to first and second elements does not mean that only two elements may be employed there or that the first element must precede the second element in some manner. Also, unless stated otherwise a set of elements may comprise one or more elements. In addition, terminology of the form “at least one of A, B, or C” or “one or more of A, B, or C” or “at least one of the group consisting of A, B, and C” used in the description or the claims means “A or B or C or any combination of these elements.” For example, this terminology may include A, or B, or C, or A and B, or A and C, or A and B and C, or 2A, or 2B, or 2C, and so on.

As used herein, the term “determining” encompasses a wide variety of actions. For example, “determining” may include calculating, computing, processing, deriving, investigating, looking up (e.g., looking up in a table, a database or another data structure), ascertaining, and the like. Also, “determining” may include receiving (e.g., receiving information), accessing (e.g., accessing data in a memory), and the like. Also, “determining” may include resolving, selecting, choosing, establishing, and the like.

While certain embodiments have been described above in detail and shown in the accompanying drawings, it is to be understood that such embodiments are merely illustrative of and not restrictive of the teachings herein. In particular, it should be recognized that the teachings herein apply to a wide variety of apparatuses and methods. It will thus be recognized that various modifications may be made to the illustrated embodiments or other embodiments, without departing from the broad scope thereof. In view of the above, it will be understood that the teachings herein are intended to cover any changes, adaptations or modifications that are within the scope of the disclosure.

1. A leadless intra-cardiac medical device, comprising:
a housing comprising an exterior surface and defining an interior space;
an inductor-capacitor resonant circuit located within the interior space defined by the housing, wherein the inductor-capacitor circuit comprises an inductive circuit and a flexible capacitive circuit electrically coupled in parallel;
a flexible material located adjacent the exterior surface of the housing and engaged with the flexible capacitive circuit to couple pressure waves to the flexible capacitive circuit;
at least one electrode located adjacent the exterior surface of the housing; and
a circuit located within the interior space defined by the housing and electrically coupled to the inductor-capacitor resonant circuit and to the at least one electrode, wherein the circuit is configured to:
process signals received from the inductor-capacitor resonant circuit to generate data representative of the pressure waves,
generate cardiac stimulation pulses for application via the at least one electrode based on control information, and
communicate via radiofrequency signaling with an external device to send the data to the external device and to receive the control information from the external device.

2. The device of claim 1, wherein the processing of the signals received from the inductor-capacitor resonant circuit to generate the data comprises:
processing the received signals to determine at least one frequency of the signals; and
determining at least one pressure value based on the determined at least one frequency, wherein the generated data comprises the determined at least one pressure value.

3. The device of claim 1, wherein the circuit is further configured to:
receive a message via radiofrequency signaling; and
commence the generation of the data as a result of the receipt of the message.

4. The device of claim 3, wherein:
the device further comprises a battery circuit;
the inductor-capacitor resonant circuit further comprises an excitation circuit electrically coupled to receive power from the battery circuit and configured to excite the inductor-capacitor resonant circuit by generating an excitation signal; and
the circuit is further configured to trigger excitation of the inductor-capacitor resonant circuit by the excitation circuit as a result of the receipt of the message.

5. The device of claim 4, wherein the circuit is further configured to receive damping signals from the inductor-capacitor resonant circuit as a result of the receipt of the message.

6. The device of claim 4, wherein the excitation circuit comprises an oscillator circuit coupled to receive power from the battery circuit.

7. The device of claim 4, wherein the excitation signal comprises at least one pulse signal or the excitation signal comprises a modulated oscillating signal having a center frequency approximately equal to a resonant frequency of the inductor-capacitor resonant circuit.

8. The device of claim 1, wherein the flexible material comprises an insulating material.

9. The device of claim 1, wherein the circuit is further configured to:
receive a signal via radiofrequency signaling; and
commence the generation of the data as a result of the receipt of the signal via radiofrequency signaling.

10. (canceled)

11. The device of claim 1, wherein:
the processing of the signals received from the inductor-capacitor resonant circuit to generate the data comprises: processing the received signals to determine at least one frequency of the signals, and determining at least one pressure value based on the determined at least one frequency, wherein the generated data comprises the determined at least one pressure value;
the circuit is further configured to receive a message via radiofrequency signaling and commence the generation of the data as a result of the receipt of the message;
the device further comprises a battery circuit;
the inductor-capacitor resonant circuit comprises an excitation circuit electrically coupled to receive power from the battery circuit and configured to excite the inductor-capacitor resonant circuit by generating an excitation signal; and
the circuit is further configured to trigger excitation of the inductor-capacitor resonant circuit by the excitation circuit as a result of the receipt of the message.

12. The device of claim 11, wherein the excitation circuit comprises an oscillator circuit coupled to receive power from the battery circuit.

13. The device of claim 1, wherein:
the processing of the signals received from the inductor-capacitor resonant circuit to generate the data comprises: processing the received signals to determine at least one frequency of the signals, and determining at least one pressure value based on the determined at least one frequency, wherein the generated data comprises the determined at least one pressure value; and
the circuit is further configured to receive a signal via radiofrequency signaling and commence the generation of the data as a result of the receipt of the signal via radiofrequency signaling.

14. The device of claim 1, wherein:
the device further comprises a battery located within the interior space defined by the housing; and
wherein the flexible capacitive circuit includes a cylindrical plate and wherein at least a portion of the cylindrical plate of the flexible capacitive circuit circumscribes at least a portion of the battery.

15. The device of claim 14, wherein the housing is hermetically sealed.

16. The device of claim 1, wherein a resonant frequency of the inductor-capacitor resonant circuit is less than 10 MHz.

17. The device of claim 1, wherein the flexible capacitive circuit includes a cylindrical plate and wherein a diameter of the cylindrical plate of the flexible capacitive circuit is greater than 7 french.

18. The device of claim 1, wherein the inductive circuit includes a coil and wherein a diameter of a coil of the inductive circuit is greater than 7 french.

19. A pressure sensing method, comprising:
triggering an excitation circuit of a leadless intra-cardiac medical device to excite an inductor-capacitor resonant circuit of the leadless intra-cardiac medical device, wherein a flexible material located adjacent an exterior surface of a housing is engaged with a capacitive circuit of the inductor-capacitor resonant circuit to couple pressure waves to the capacitive circuit;
processing signals produced by the excited inductor-capacitor resonant circuit to determine at least one frequency of the signals;
generating data representative of pressure external to the leadless intra-cardiac medical device based on the determined at least one frequency; and
transmitting the generated data via radiofrequency signaling to an external device.

20. The method of claim 19, further comprising:
receiving a message via radiofrequency signaling; and
commencing the generation of the data as a result of the receipt of the message.

21. The method of claim 20, wherein the triggering of the excitation circuit is based on the receipt of the message.
